(19) United States
(12) Patent Application Publication
(10) Pub. No.: US 2025/0216390 A1
(43) Pub. Date: Jul. 3, 2025

(54) Rolling Sensor Systems for Detecting Analytes and Diagnostic Methods Related Thereto

(71) Applicant: Emory University, Atlanta, GA (US)

(72) Inventors: Selma Piranej, Atlanta, GA (US); Khalid Salaita, Atlanta, GA (US); Alisina Bazrafshan, San Diego, CA (US)

(21) Appl. No.: 18/853,553

(22) PCT Filed: Mar. 28, 2023
(86) PCT No.: PCT/US2023/065070

§ 371 Date: Oct. 2, 2024

Related U.S. Application Data
(60) Provisional application No. 63/325,775, filed on Mar. 31, 2022.

(51) Int. Cl. G01N 33/569; G01N 33/53; G01N 33/543; G06V 10/75; G06V 20/40; G06V 20/69

(52) U.S. Cl. CPC G01N 33/56983; G01N 33/5308; G01N 33/54313; G06V 10/751; G06V 20/40; G06V 20/698; G01N 2333/11; G01N 2333/165; G06V 2201/04

(57) ABSTRACT

This disclosure relates to sensing the movement of DNA rolling motors comprising microparticles or rods on transduction material in the presence of viruses, microbes, or other analytes for diagnostic testing. In certain embodiments, the presence of viral particles, other target microbial biomolecules, or analytes stall the motor by specifically binding to aptamers crosslinking the analytes to the particles, rods, or surface of the transduction material. It is contemplated that microparticles or other rolling motors move along a surface whereby an aptamer targets an analyte, e.g., viral SARS-CoV-2, and acts to inhibit, reduce, or restrict the speed, acceleration, or area of movement on the surface indicating the presence of the analyte in the sample.

### CROSS-REFERENCE TO RELATED APPLICATIONS

This application claims the benefit of U.S. Provisional Application No. 63/325,775 filed Mar. 31, 2022. The entirety of this application is hereby incorporated by reference for all purposes.

### STATEMENT REGARDING FEDERALLY SPONSORED RESEARCH OR DEVELOPMENT

This invention was made with government support under 1905947 awarded by the National Science Foundation and AA029345 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22100PCT.xml. The XML file is 18 KB, was created on Mar. 28, 2023, and is being submitted electronically via the USPTO patent electronic filing system.

### BACKGROUND

Many common colds are due to certain coronavirus (CoV) strains associated with mild symptoms. More dangerous human strains include severe acute respiratory syndrome associated coronavirus (SARS-CoV-1) and SARS-CoV-2 (also referred to as COVID-19). In humans, SARS-CoV-2 can be transferred from individuals who have mild symptoms or are asymptomatic and has caused numerous deaths worldwide. Thus, there is a need to find effective methods for early detection.

Yehl et al. report high-speed DNA-based rolling motors powered by RNase H. Nat Nanotechnol, 2016, 11 (2): 184-90. See also U.S. Pat. No. 10,738,349.

Bazrafshan et al. report DNA gold nanoparticle motors demonstrate processive motion with bursts of speed up to 50 nm per second. ACS Nano, 2021, 15, 8427-8438.

Chakraborty et al. report aptamers for viral detection and inhibition. ACS Infect Dis, 2022, 8, 4, 667-692.

EP Pat. 2255015 (2015) reports methods for assembly of DNA aptamer bead conjugates for use sandwich assays.

References cited herein are not an admission of prior art.

### SUMMARY

This disclosure relates to sensing the movement of DNA rolling motors comprising microparticles or rods on transduction material in the presence of viruses, microbes, or other analytes for diagnostic testing. In certain embodiments, the presence of viral particles, other target microbial biomolecules, or analytes stall the motor by specifically binding to aptamers crosslinking the analytes to the particles, rods, or surface of the transduction material. It is contemplated that microparticles or other rolling motors move along a surface whereby an aptamer targets an analyte, e.g., viral SARS-CoV-2, and acts to inhibit, reduce, or restrict the speed, acceleration, or area of movement on the surface indicating the presence of the analyte in the sample.

In certain embodiments, the presence of viral particles, other target microbial biomolecules, or analytes stall the motor by specifically binding to aptamers crosslinking the analytes to the surface of the particle, rod, and/or the surface.

In certain embodiments, the motors are coated with single stranded segments of DNA, RNA, and aptamers that bind viruses, microbes, microbial specific biomolecules, or other analytes. It is contemplated that microparticles or other rolling motors move along a surface whereby an aptamer targets an analyte, e.g., coronavirus, SARS-CoV-2, SARS-CoV-1, measles, mumps, influenza, Middle East Respiratory Syndrome (MERS) virus, and act to inhibit, reduce, or restrict the speed or area of movement on the surface indicating the presence of the analyte in the sample.

In certain embodiments, this disclosure relates to methods of detecting the presence of a virus, microbe, biomolecule, ligand, or other analyte in a sample comprising contacting a sample suspected of containing a virus, microbe, biomolecule, ligand, or other analyte and a spherical particle or circular rod comprising a coating of single stranded DNA and an aptamer that specifically binds the virus, microbe, biomolecule, ligand, or other analyte providing a sample exposed spherical particle or circular rod; providing a planar substrate comprising a coating of single stranded RNA and an aptamer that specifically binds the virus, microbe, biomolecule, ligand, or other analyte; placing the sample exposed spherical particle or circular rod on the surface of the planar substrate in the presence of RNase H such that the particle or rod moves on the surface of the substrate; and measuring quantitatively the movement of the sample exposed spherical particle or circular rod on the surface of the planar substrate providing a sample exposed movement value; comparing the sample exposed movement value to a reference movement value correlated to the movement of the particle or rod on the surface of the planar substrate in the presence of RNase H and in the absence of the virus, microbe biomolecule, ligand, or other analyte, and detecting the presence of the virus, microbe biomolecule, ligand, or other analyte in a sample if the movement value is less than the reference movement value indicative of the presence of the virus, microbe biomolecule, ligand, or other analyte in a sample.

In certain embodiments, methods further comprises detecting that the particle moves at a lower velocity or speed on the substrate or at a lower displacement measured by an lesser area of movement over a surface, e.g., from a central point of origin when compared to a control particle would move in the absence of the microbe or other analyte in the sample; and correlating the velocity, speed less or lower displacement on the substrate to presence of the microbial biomolecule or other analyte in the sample thereby detecting the presence of the microbe or other analyte in the sample.

In certain embodiments, it is contemplated that detection can be accomplished without a fluorescence readout or absorbance measurement. In certain embodiments, detection of the viral target is through a change in the rate of velocity, speed, acceleration, or displacement of the motor over a set time. In certain embodiments, it is contemplated that one can multiplex and detect multiple respiratory analytes, viruses, or microbes in the same assay. In certain embodiments, binding agents are aptamers specific for viral coat proteins of a virus, coronavirus and/or influenza which are used for sensing a virus of concern, virus of interest, or community spread coronavirus or influenza virus.

In certain embodiments, this disclosure contemplates diagnostic tests, systems, and computer readable mediums comprising data generated using methods disclosed herein or instructions to capture, calculate, and generate data associated with measured parameters disclosed herein.

### BRIEF DESCRIPTION OF THE SEVERAL VIEWS OF THE DRAWINGS

FIGS. 1A-C show a contemplated method for a “Rolosense” at home assay.

FIG. 1A illustrates that nasal or saliva samples are collected and contacted with the DNA micromotor particles (about 5 micron beads).

FIG. 1B illustrates micromotors (beads) modified with aptamers against SARS-CoV-2 viral particles. Because the chip is also modified with SARS-CoV-2 aptamers, the presence of virus particles leads to motor stalling. Motion is recorded using a video camera.

FIG. 1C illustrates smart phone and software analysis provide test result by determining displacement.

FIG. 2A illustrates Rolosensors which are chemical-to-mechanical signal transducers that use a mechanism of DNA motor motion.

FIG. 2B shows a representative brightfield (BF) black and white image and trajectory (line) from a timelapse movie that tracked a single microparticle 30 minutes after the RNase H addition. The same region was then imaged in a Cy3 fluorescence channel to reveal the location of depleted Cy3 signal.

FIG. 2C shows data on the log mean squared displacement (MSD) versus time analysis from individual particle trajectories, which is shown with circles and plotted on a logarithmic scale. The line indicates the average slope derived from all the individual particle trajectories.

FIG. 3A shows a schematic illustrating a DNA motor and chip functionalization. The DNA motors were modified with a binary mixture of with DNA leg and aptamers that have high affinity for SARS-CoV-2 spike protein. The Rolosense chip is a gold film also comprised of two nucleic acids: the RNA/DNA chimera, which is referred to as the RNA fuel, and the same aptamer as the motor.

FIG. 3B shows a schematic illustrating the detection of SARS-CoV-2 virus. In the presence of VLPs expressed with spike protein (spike VLPs), the motors stall on the Rolosense chip following the addition of the RNase H enzyme as the stalling force (arrow) is greater than the force generated by the motor (arrow). When incubated with the bald VLPs, or VLPs lacking the spike protein, the motors respond with motion and roll on the chip in the presence of RNase H.

FIG. 3C shows data on net displacement of over 100 motors incubated with 25 pM bald and spike VLPs. Experiments were performed in triplicate.

FIG. 3D shows a plot of data indicating the difference in net displacement between the bald/spike VLPs normalized by the bald VLP displacement in conditions using different aptamers.

FIG. 4 shows data detecting SARS-CoV-2 WA-1 and B.1.617.2 using smartphone readout. A cellphone microscope (Cellscope™) is 3D printed and includes an LED flashlight along with a smartphone holder and simple optics. A microscopy image shows DNA motors that were analyzed using a particle tracking analysis software. Moving particles show a color trail that indicates position over time (0 to 30 min). The diameter of the motors is 5 μm. Plots show data of net displacement of motors incubated with different concentrations of UV-inactivated SARS-CoV-2 Washington WA-1 and B.1.617.2 samples spiked in artificial saliva. The net displacement of the motors was calculated from 15-min videos acquired using a cellphone camera. The motors were functionalized with aptamer 3.

FIG. 5A illustrates detecting SARS-CoV-2 virus in breath condensate. Schematic of breath condensate sample collection and incubation of DNA motors with spiked-in virus particles. Fluorescence and brightfield imaging were done using aptamer 3 modified DNA motors without virus, with 10⁷ copies/mL of SARS-CoV-2 B.1.617.2. Fluorescence and brightfield imaging were done using aptamer 4 modified DNA motors without virus, with 10⁷ copies/mL of SARS-CoV-2 BA.1. Samples without virus show long depletion tracks in the Cy3-RNA channel but no tracks are observed following sample incubation with 10⁷ copies/mL of SARS-CoV-2 B.1.617.2 and BA.1.

FIG. 5B shows data from plots of net displacement of over 300 motors with no virus and different concentrations of UV-inactivated SARS-CoV-2 B.1.617.2, and BA.1. UV-inactivated SARS-CoV-2 samples were spiked in breath condensate and incubated with the motors functionalized with aptamer 3 (B.1.617.2) and aptamer 4 (BA.1) at room temperature.

### DETAILED DISCUSSION

Before the present disclosure is described in greater detail, it is to be understood that this disclosure is not limited to embodiments described, and as such may, of course, vary. An “embodiment” refers to an example and is not necessarily limited to such example. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps. The term “comprising” in reference to an oligonucleotide having a nucleic acid sequence refers to an oligonucleotide or peptide that may contain additional 5′ (5′ terminal end) or 3′ (3′ terminal end) nucleotides or N- or C-terminal amino acids, i.e., the term is intended to include the oligonucleotide sequence or peptide sequence within a larger nucleic acid or peptide. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein. The term “consisting of” in reference to an oligonucleotide or peptide having a nucleotide or peptide sequence refers an oligonucleotide or peptide having the exact number of nucleotides or amino acids in the sequence and not more or having not more than a range of nucleotide expressly specified in the claim. For example, “5′ sequence consisting of” is limited only to the 5′ end, i.e., the 3′ end may contain additional nucleotides. Similarly, a “3′ sequence consisting of” is limited only to the 3′ end, and the 5′ end may contain additional nucleotides.

As used herein, the term “about” or “approximately” refers to plus or minus 10 or 20 percent of the recited value, so that, for example, “about 0.125” means 0.125 plus/minus 0.025, and “about 1.0” means 1.0 plus/minus 0.2.

The term “sample” is used in its broadest sense, in that it has chemical makeup that is physical for analysis, i.e., analyte. In one sense it can refer to a nasal fluid, saliva, cough droplets, or expelled droplets of saliva into the air, e.g., produced by speaking, or other lung fluid blood. In another sense, it is meant to include a specimen or culture obtained from any source, as well as biological and environmental samples. Biological samples include bodily fluids, urine, feces, nasal drip, seminal fluid, hair, skin (dead or epithelial layer of skin), finger or toenail clipping, and blood products such as plasma, serum, and the like. Environmental samples include environmental material such as surface matter, soil, water, crystals, and industrial samples. Preferably the sample is from a subject and encompass fluids, solids, tissues, and gases. As used herein, “subject” refers to any animal, preferably a human patient, livestock, or domestic pet.

As used herein, the terms “aptamer” refers to an oligonucleotide or nucleobase polymer that specifically binds to a target molecule, e.g., protein. Aptamers can be comprised of RNA or DNA or chemically modified forms. In some embodiments, the aptamer binds to a specific region or amino acid sequence of a target protein. The aptamers typically have secondary structures such as hairpins or stem-loop structures but can contain linear segments. Aptamers can be structure-switching aptamers. The Systematic Evolution of Ligands by EXponential enrichment method, or SELEX, is a combinatorial chemistry technique for producing oligonucleotides of either single-stranded DNA or RNA that specifically bind to one or more target ligands. The method involves selection from a mixture of candidate oligonucleotides and step-wise iterations of binding, partitioning and amplification, using the same general selection scheme, to achieve a desired level of binding affinity and selectivity. SELEX has been used to evolve nucleic acid aptamers of extremely high binding affinity to a variety of targets. Some of these targets include, for example, viruses. See Darfeuille et al. Biochemistry, 2006, 45: 12076-12082. Other examples of aptamers and methods of selection (design) can be found, for instance, in U.S. Pat. No. 5,693,502 and U.S. Pub. Pat. App. No. 2014/0342918, incorporated herein by reference.

The term “specific binding agent” refers to a molecule, such as a protein, antibody, or nucleic acid, that binds a target molecule with a greater affinity than other random molecules, proteins, or nucleic acids. Examples of specific binding agents include antibodies that bind an epitope of an antigen or a receptor which binds a ligand. “Specifically binds” refers to the ability of a specific binding agent (such as an ligand, receptor, enzyme, nucleic acid, antibody or binding region/fragment thereof) to recognize and bind a target molecule such that its affinity (as determined by, e.g., affinity ELISA or other assays) is at least 10 times as great, but optionally 50 times as great, 100, 250 or 500 times as great, or even at least 1000 times as great or more as the affinity of the same for any other random molecule, nucleic acid, or polypeptide.

As used herein, the term “ligand” refers to an organic molecule, i.e., substantially comprised of carbon, hydrogen, and oxygen, that binds a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin may bind the glycan. As the glycan is typically smaller and surrounded by the lectin during binding, it may be considered a ligand even though it is a receptor of the lectin binding signal on the cell surface. In another example, a double stranded oligonucleotide sequence contains two complimentary nucleic acid sequences. Either of the single stranded sequences may be consider the ligand or receptor of the other. In certain embodiments, a ligand is contemplated to be a compound that has a molecular weight of less than 500 or 1,000. In certain embodiments, a receptor is contemplated to be a compound that has a molecular weight of greater than 2,000 or 5,000. In any of the embodiments disclosed herein the position of a ligand and a receptor may be switched.

As used herein, the term “surface” refers to the outside part of an object. The area is typically of greater than about one hundred square nanometers, one square micrometer, or more than one square millimeter. Examples of contemplated surfaces are on a particle, bead, wafer, array, well, microscope slide, polymer (plastic), metal, or transparent or opaque glass or other material.

The term “conjugated” refers to linking molecular entities through covalent bonds, or by other specific binding interactions, such as due to hydrogen bonding or other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to restrict the breaking of bonds in order to implement the intended results.

A “linking group” refers to any variety of molecular arrangements that can be used to bridge or conjugate molecular moieties together. An example formula may be-Rn-wherein R is selected individually and independently at each occurrence as: —CRₙRₙ—, —CHRₙ—, —CH, —C—, —CH₂—, —C(OH)Rₙ, —C(OH)(OH)—, —C(OH)H, —C(Hal)Rₙ—, —C(Hal)(Hal)—, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH—CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, polyethylene glycols, amides, esters, and aromatic groups.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as 35S or 1311), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

A “fluorescent tag” or “fluorescent dye” refers to a compound that can re-emit electromagnetic radiation upon excitation with electromagnetic radiation (e.g., ultraviolet light) of a different wavelength. Typically, the emitted light has a longer wavelength (e.g., in visible spectrum) than the absorbed radiation. As the emitted light typically occurs almost simultaneously, i.e., in less than one second, when the absorbed radiation is in the invisible ultraviolet region of the spectrum, the emitted light may be in the visible region resulting in a distinctive identifiable color signal. Small molecule fluorescent tags typically contain several combined aromatic groups, or planar or cyclic molecules with multiple interconnected double bonds. Chen et al. report a variety of fluorescent tags that can be viewed across the visible spectrum. Nature Biotechnology, 2019, 37, 1287-1293. The term “fluorescent tag” is intended to include compounds of larger molecular weight such as natural fluorescent proteins, e.g., green fluorescent protein (GFP) and phycobiliproteins (PE, APC), and fluorescence particles such as quantum dots, e.g., preferably having 2-10 nm diameter.

The terms, “nucleic acid,” or “oligonucleotide,” is meant to include nucleic acids, ribonucleic or deoxyribonucleic acid, mixtures, nucleobase polymers, or analog thereof. An oligonucleotide can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine.

The term “nucleobase polymer” refers to nucleic acids and chemically modified forms with nucleobase monomers. In certain embodiments, methods and compositions disclosed herein may be implemented with nucleobase polymers comprising units of a ribose, 2′deoxyribose, locked nucleic acids (1-(hydroxymethyl)-2,5-dioxabicyclo[2.2.1]heptan-7-ol), 2′-O-methyl groups, a 3′-3′-inverted thymidine, phosphorothioate linkages, or combinations thereof. In certain embodiments, the nucleobase polymer may be less than 100, 50, or 35 nucleotides or nucleobases.

Nucleobase monomers are nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may be single or double stranded or both, e.g., they may contain overhangs. Nucleobase polymers may contain naturally occurring or synthetically modified bases and backbones. In certain embodiments, a nucleobase polymer need not be entirely complementary, e.g., may contain one or more insertions, deletions, or be in a hairpin structure provided that there is sufficient selective binding.

Regarding the nucleobases, it is contemplated that the term encompasses isobases, otherwise known as modified bases, e.g., are isoelectronic or have other substitutes configured to mimic naturally occurring hydrogen bonding base-pairs, e.g., within any of the sequences herein U may be substituted for T, or T may be substituted for U. Examples of nucleotides with modified adenosine or guanosine include, but are not limited to, hypoxanthine, xanthine, 7-methylguanine. Examples of nucleotides with modified cytidine, thymidine, or uridine include 5,6-dihydrouracil, 5-methylcytosine, 5-hydroxymethylcytosine. Contemplated isobases include 2′-deoxy-5-methylisocytidine (iC) and 2′-deoxy-isoguanosine (iG) (see U.S. Pat. Nos. 6,001,983; 6,037,120; 6,617,106; and 6,977,161).

Nucleobase polymers may be chemically modified, e.g., within the sugar backbone or on the 5′ or 3′ ends. As such, in certain embodiments, nucleobase polymers disclosed herein may contain monomers of phosphodiester, phosphorothioate, methylphosphonate, phosphorodiamidate, piperazine phosphorodiamidate, ribose, 2′-O-methy ribose, 2′-O-methoxyethyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphon amidate, morpholin-2-ylmethanol, (2-(hydroxymethyl)morpholino) (piperazin-1-yl)phosphinate, or peptide nucleic acids or combinations thereof.

In certain embodiments, the nucleobase polymer can be modified to contain a phosphodiester bond, methylphosphonate bond or phosphorothioate bond. The nucleobase polymers can be modified, for example, 2′-amino, 2′-C-allyl, 2′-fluoro, 2′-O-methyl, 2′-H of the ribose ring. Constructs can be purified by gel electrophoresis using general methods or can be purified by high pressure liquid chromatography and re-suspended in water.

In certain embodiments, nucleobase polymers include one or more (e.g., about 1, 2, 3, 4, 5, 6, 7, 8, 9, 10, or more) LNA “locked nucleic acid” nucleotides such as a 2′,4′-C methylene bicyclo nucleotide (see for example U.S. Pat. Nos. 6,639,059, 6,670,461, 7,053,207).

In certain embodiments, the disclosure features modified nucleobase polymers, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating,” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any programming language. It will be appreciated that a variety of programming languages may be used to implement embodiments of the disclosure.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general-purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

### Compositions and Methods of Use

In certain embodiments, this disclosure relates to sensing the movement of DNA rolling motors, microparticles or rods, on transduction material in the presence of a sample for detection of an analyte, e.g., viruses, microbes. In certain embodiments, the presence of viral particles, microbes, biomolecules, or other analytes stall the motor by specifically binding to aptamers on the particles and/or aptamers on the planar surface crosslinking the particle to the particles, surface, or both. In certain embodiments, the motors are coated with single stranded segments of RNA, single stranded segments of DNA, and aptamers that bind viral or other microbial specific biomolecules or analytes. In certain embodiments, it is contemplated that microparticles or other rolling motors, move along a surface whereby an aptamer targets SARS-CoV-2 or other viral derived biomolecule and acts to inhibit or reduces the velocity, speed, acceleration, or movement area on the surface.

In certain embodiments, this disclosure relates to methods of detecting the presence of a virus, microbe, biomolecule, ligand, or other analyte in a sample comprising contacting a sample suspected of containing a virus, microbe, biomolecule, ligand, or other analyte and a spherical particle or circular rod comprising a coating of single stranded DNA and an aptamer that specifically binds the virus, microbe biomolecule, ligand, or other analyte providing a sample exposed spherical particle; providing a planar substrate comprising a coating of single stranded RNA and an aptamer that specifically binds the virus, microbe, biomolecule, ligand, or other analyte; placing the sample exposed spherical particle or circular rod on the surface of the planar substrate in the presence of RNase H such that the particle moves on the surface of the substrate; and measuring quantitatively the movement of the sample exposed spherical particle or circular rod on the surface of the planar substrate providing a sample exposed movement value; comparing the sample exposed movement value to a reference movement value correlated to the movement of the spherical particle on the surface of the planar substrate in the presence of RNase H and in the absence of the virus, microbe, biomolecule, ligand, or other analyte, and detecting the presence of the virus, microbe, biomolecule, ligand, or other analyte in a sample if the movement value associated with the presence of an analyte in the sample, e.g., is less than the reference movement value.

In certain embodiments, this disclosure relates to methods of detecting the presence of the virus, microbe, biomolecule, ligand, or other analyte in a sample if the movement value is substantially less than as the reference movement value, i.e., the reference value is a normal value for movement of the spherical particle or circular rod in the absence of the analyte.

In certain embodiments, this disclosure relates to methods of detecting the absence of the virus, microbe, biomolecule, ligand, or other analyte in a sample if the movement value is substantially the same as the reference movement value, i.e., the reference value is a normal value for movement of the spherical particle or circular rod in the absence of the analyte.

In certain embodiments, this disclosure relates to methods of detecting the presence of the virus, microbe, biomolecule, ligand, or other analyte in a sample if the movement value is substantially the same as or less than the reference movement value, i.e., the reference value is a threshold value for movement of the spherical particle or circular rod in the presence of the analyte.

In certain embodiments, this disclosure relates to methods of detecting the absence of the virus, microbe, biomolecule, ligand, or other analyte in a sample if the movement value is greater the reference movement value, i.e., the reference value is a threshold value or normal value for movement of the spherical particle or circular rod in the presence of the analyte.

In certain embodiments, methods further comprises detecting that the particle moves at a velocity, acceleration, or speed less on the substrate or at a lower displacement measured by an lesser area of movement over a surface, e.g., from a central point of origin when compared to a control particle or rod would move in the absence of the microbe or other analyte in the sample; and correlating the reduced velocity, speed, acceleration, or lower displacement on the substrate to presence of the microbe, biomolecule, or other analyte in the sample thereby detecting the presence of the microbe or other analyte in the sample.

In certain embodiments, it is contemplated that detection can be accomplished without a fluorescence readout or absorbance measurements, e.g., utilizing brightfield measurements using a camera, video camera, or microscope. In certain embodiments, detection of the viral target or other analyte is through reduced velocity, speed, acceleration, or motion of the motor or reduce displacement over an area as detected over time. In certain embodiments, it is contemplated that one can multiplex and detect multiple analytes, e.g., respiratory viruses or microbes in the same assay with multiple zones, or areas on the same planar surfaces (chips, wells, lanes). In certain embodiments, binding agents are aptamers specific for viral coat proteins, spike proteins, or capsid proteins of a virus, coronavirus and/or influenza virus which are used for detecting a virus of concern, virus of interest, community spread, coronavirus virus, influenza, or other virus.

In certain embodiments, this disclosure contemplates diagnostic tests, systems, computers, and computer readable mediums comprising data generated using methods disclosed herein or instructions to capture, calculate, and generate data as disclosed herein.

In certain embodiments, this disclosure methods of detecting the presence of a microbe, a biomolecule, ligand, or other analyte in a sample obtained from a subject comprising, providing a sample and a rolling particle system, wherein the sample comprises a microbial biomolecule associated with the microbe or other analyte, wherein the rolling particle system comprises, a spherical particle or circular rod comprising a coating of single stranded DNA and a specific binding agent, e.g., an aptamer, that specifically binds the microbial biomolecule or other analyte; a planar substrate comprising a coating of single stranded RNA and an aptamer that specifically binds the microbial biomolecule or other analyte; contacting the spherical particle with the sample providing a particle aptamer bound to the microbial biomolecule or other analyte; placing the particle aptamer bound to the microbial biomolecule or other analyte on the surface of the planar substrate in the presence of RNase H such that the particle moves on the surface of the substrate at a velocity, acceleration, or speed less or at lower displacement than the particle would move in the absence of the microbial biomolecule or other analyte in the sample; detecting that the particle moves at a reduced velocity, acceleration, or speed on the substrate or at a lower displacement measured by an lesser area of movement over a surface from a central point of origin when compared to a control particle would move in the absence of the microbial biomolecule or other analyte in the sample; and correlating the reduced speed (lower speed or acceleration) or lower displacement on the substrate to presence of the microbial biomolecule or other analyte in the sample thereby detecting the presence of the microbe or other analyte in the sample.

In certain embodiments, the molar ratio of the specific binding agent or aptamer to the single stranded DNA coated on the spherical particle or circular rod is about 1:10 or less, or the specific binding agent or aptamer is about 10% by weight.

In certain embodiments, the molar ratio of specific binding agent or aptamer to the single stranded RNA coated on the planar substrate is about 1:1 or less, or the specific binding agent or aptamer is about 50% by weight.

In certain embodiments, the aptamer binds a coronavirus spike protein or ACE2 receptor binding domain.

In certain embodiments, the aptamer comprises the nucleobase sequence as disclosed herein, e.g., of aptamer 1,

(SEQ ID NO: 14)
CGCGGTCATTGTGCATCCTGACTGACCCTAAGGTGCGAACATCGC
CCGCG,
aptamer 2,
(SEQ ID NO: 15)
GGAGAGGAGGGAGATAGATATCAACCCATGGTAGGTATTGCTTGG
TAGGGATAGTGGGCTTGATGTTTCGTGGATGCCACAGGAC,
aptamer 3,
(SEQ ID NO: 16)
CCCATGGTAGGTATTGCTTGGTAGGGATAGTGGG,
or
aptamer 4,
(SEQ ID NO: 17)
ACGCCAAGGTGTCACTCCGTAGGGTTTGGCTCCGGGCCTGGCGTC
GGTCGCGAAGCATCTCCTTGGCGT,
influenza aptamer, or
(SEQ ID NO: 18)
GGCAGGAAGACAAACAGCCAGCGTGACAGCGACGCGTAGGGACCG
GCATCCGCGGGTGGTCTGTGGTGCTGTGCA.

In certain embodiments, this disclosure contemplates a specific binding agent, e.g., an aptamer, disclosed herein conjugated to a spherical particle, circular rod, a planar substrate, a label, or fluorescent dye.

In certain embodiments, the microbe is a virus or bacteria. In certain embodiments, the microbe is a coronavirus. In certain embodiments, the microbe is SARS-CoV-2. In certain embodiments, the microbe is an influenza virus.

In certain embodiments, detecting changes in particle movement, e.g., the particle moves at a rate on the surface of the substrate that is less than the rate the particle would move in the absence of the microbial biomolecule or other analyte in the sample, is by use of a camera or video camera recording and the picture or video recording is analyzed by a computer to provide a quantitative estimation(s) of displacement, velocity, acceleration, or speed.

In certain embodiments, detecting that the particle moves on the surface of the substrate at a lower displacement or in a more restricted area than the particle would move in the absence of the microbial biomolecule or other analyte in the sample is by use of a video camera such as a smart phone.

In certain embodiments, the RNA density on the planar substrate is between about 2×10⁴ molecules molecules/μm² and about 6×10⁴ molecules/μm².

In certain embodiments, the spherical particle or circular rod has a diameter of 0.001 micrometers to 1 centimeter, or 0.01 to 10 micrometers, or 1 to 5 micrometers.

In certain embodiments, the DNA on the spherical particle has a density coverage of about 50,000 or 90,000 molecules/μm² or more.

In certain embodiments, the RNase H is at a concentration of 100 or 140 nM or more, e.g., up to a 10-fold increase thereof.

In certain embodiments, this disclosure relates to rolling particle systems comprising, a spherical particle or circular rod comprising a coating of single stranded DNA and a specific binding agent, e.g., an aptamer that specifically binds the microbial biomolecule or other analyte; and a planar substrate comprising a coating of single stranded RNA and an aptamer that specifically binds the microbial biomolecule or other analyte.

In certain embodiments, the molar ratio of the specific binding agent or aptamer to the single stranded DNA coated on the spherical particle or circular rod is about 1:10 or less, or the specific binding agent or aptamer is about 10% by weight.

In certain embodiments, the molar ratio of specific binding agent or aptamer to the single stranded RNA coated on the planar substrate is about 1:1 or less, or the specific binding agent or aptamer is about 50% by weight.

In certain embodiments, the RNA density on the planar substrate is between about 2×10⁴ molecules molecules/μm² and about 6×10⁴ molecules/μm².

In certain embodiments, the spherical particle has a diameter of 0.001 micrometers to 1 centimeter, or 0.01 to 10 micrometers, or 1 to 5 micrometers.

In certain embodiments, the DNA on the spherical particle has a density coverage of about 50,000 or 90,000 molecules/μm² or more.

In certain embodiments, the RNase H is at a concentration of 100 or 140 nM or more, e.g., up to a 10-fold increase thereof.

In certain embodiments, the aptamer comprises the nucleobase sequence as disclosed herein, e.g., of aptamer 1,

(SEQ ID NO: 14)
CGCGGTCATTGTGCATCCTGACTGACCCTAAGGTGCGAACATCGC
CCGCG,
aptamer 2,
(SEQ ID NO: 15)
GGAGAGGAGGGAGATAGATATCAACCCATGGTAGGTATTGCTTGG
TAGGGATAGTGGGCTTGATGTTTCGTGGATGCCACAGGAC,
aptamer 3,
(SEQ ID NO: 16)
CCCATGGTAGGTATTGCTTGGTAGGGATAGTGGG,
or
aptamer 4,
(SEQ ID NO: 17)
ACGCCAAGGTGTCACTCCGTAGGGTTTGGCTCCGGGCCTGGCGTC
GGTCGCGAAGCATCTCCTTGGCGT,
influenza aptamer, or
(SEQ ID NO: 18)
GGCAGGAAGACAAACAGCCAGCGTGACAGCGACGCGTAGGGACCG
GCATCCGCGGGTGGTCTGTGGTGCTGTGCA.

### Home Testing for SARS-CoV-2

Serological, antigen, and PCR based assays have increased the diversity and availability of testing for SARS-CoV-2. However, rapid spread of additional waves of covid-19 continue. Accordingly, developing tools for rapid at home detection of SARS-CoV-2, its variants, and other viral targets are needed as it has the potential to complement the suite of vaccines to help protect the US and world from future emerging threats. The SARS-CoV-2 virus is not the only threat, and many other viruses including measles, mumps, influenza, MERS and SARS-CoV cause severe acute respiratory syndrome, and Middle East respiratory syndrome, share many symptoms with SARS-CoV-2.

It is contemplated that devices and methods disclosed herein can be used in the detection of SARS-CoV-2, its variants, and other respiratory viruses in parallel and in real time (FIG. 1A-C). Testing is performed at home producing the result in less than 60 min. A DNA micromotor acts as the molecular transducer that can be detected by a disposable microfluidic device that is Wi-Fi-enabled providing both geographical tracing and rapid detection. In this assay, a DNA microparticle “motor” consumes chemical energy in an RNA chip to generate mechanical work. Microparticle motors achieve velocities of 5 μm/min and translocate distances up to 10-3 meters, approaching the capabilities of natural motor proteins (1 μm/s and 10⁻³ m). See Yehl et al. High-speed DNA-based rolling motors powered by RNase H. Nat Nanotechnol, 2016, 11(2):184-90. See also U.S. Pat. No. 10,738,349.

These are fast and processive synthetic motors. These motors can run continuously for up to 24 hrs. The motor consists of a 5 μm diameter DNA-coated silica particle that hybridizes to a surface modified with complementary RNA. The particle moves upon addition of ribonuclease H (RNase H), which selectively hydrolyzes hybridized RNA but not single stranded RNA (FIG. 2A). Since the driving force for movement is derived from the free energy of binding new single stranded RNA that biases motion away from consumed substrate (FIG. 2B). This type of motion is classified as a burnt-bridge mechanism of motion. The spherical geometry allows for rolling (hence the name “Rolosense”), which is a fundamentally different mode for translocation. Particle motion can be recorded and tracked using a smartphone. This makes these motors an attractive signal transducer to detect the SARS-CoV-2 virus and to convert this molecular binding event into an electronic signal readily traced in space and time (FIG. 2C).

Smartphone integration is contemplated. Due to the micron-sized moving particle and large distances travelled, a smartphone camera with a lens can be used for readout or a high-end optical microscope. Plastic lens mounted onto the camera of a smartphone were used to visualize motor particles. Using the smart phone readout, one can detect a single nucleotide mutation (SNP) using the net displacement of the motor over a 15 min duration. The readout required 15 min and did not use amplification or fluorescence. These motors can also be detected using an ESP32-CAM microprocessor.

One can use of an ESP32-CAM module for readout of the assay on a custom multichannel microfluidic chip. ESP32-CAM is a video camera with WiFi/Bluetooth connectivity, a CPU, and provides image streaming at different levels of optical resolution. Rolosense assays may employed smart phone cameras for readout. However, the ESP32-CAM offers a more compact designs, customized microfluidic integrated cartridge design and is more appropriate for the home testing uses.

It is contemplated that one can use a DNA microparticle motor as a virus sensing and transduction material (VSTM) to report on specific molecular events. The presence of single copies of viral particle target will “stall” the motor by crosslinking the particle to the surface. In other words, the microparticle moves along the surface through a “cog-and-wheel” mechanism and only specific the SARS-CoV-2 viral target acts as a “wrench” to inhibit this activity. In certain embodiments, it is contemplated that a fluorescence readout or absorbance measurements are not needed to detect a nucleic acid. Instead, detection of the viral target is through the speed of the motor.

An advantage of Rolosense is the ability to multiplex and detect multiple respiratory viruses in the same assay. One can use aptamers specific for influenza A and integrate these into Rolosense assay and perform sensing of influenza A using conditions established for SARS-CoV-2. It is contemplated that this method minimizes false positive results due to similar symptoms. Because the motor detects the virus itself rather than the nucleic acid material, there is no need for enzymatic amplification and sample processing steps. This leads to a rapid (30 min readout) without any intervention. This approach represents a biosensor design that focuses on mechanical stability of virus binding ligands rather than the Kd of analyte binding. It is contemplated that multiple different viral target analytes and biological processes can be investigated using this motion-based sensing approach.

Rolosense is contemplated for use in SARS-CoV-2 diagnostic tests for the detection of virus particles in saliva and nasal swab samples self-collected by individuals, e.g., human patients. In certain embodiments, the patient is 18 years or older or less than 18 years old. Patients may be symptomatic or asymptomatic that suspect being exposed or infected and desire a rapid readout. A positive result is indicative of an active SARS-CoV-2 infection and may be validated through clinical testing under the supervision of a healthcare provider. The Rolosense assay was tested and optimized the using virus-like particles (VLPs) expressing the trimeric spike protein. The optimal screening conditions were then used to test UV-inactivated (Washington WA-1) strain SARS-CoV-2. The Rolosense assay is compatible with detecting authentic virus samples. The motors stall in the presence of virus as the viral particle is trapped at the motor-chip junction. In experiments a limit of detection (LOD) of about 10³ copies/mL was identified for the Washington strain (WA-1). The data suggests that micromotors stall upon encountering single virus particles. Hence, with volume miniaturization (using microfluidics), the LOD will be single virus copies. Also contemplated is the ability to multiplex by detecting influenza A and SARS-CoV-2 in the same “pot” readout via smartphone in as little as 15 mins. Overall, Rolosense enables rapid, sensitive, and multiplexed viral detection for disease monitoring.

### Rolosense: Mechanical Detection of SARS-CoV-2 Using a DNA-Based Motor

A mechanical-based detection method of SARS-CoV-2 viral particles was developed that is label-free and does not require fluorescence readout or absorbance measurements. Because the motor detects the virus itself rather than the nucleic acid material, there is typically no need for enzymatic amplification and sample processing steps. Rolosense employs a “mechanical transduction” mechanism based on performing a mechanical test of the analyte and the outcome of this mechanical test is converting viral binding into motion output. The motors typically stall if the mechanical stability of virus binding ligands, e.g., aptamers, exceed the forces generated by the motor. The aptamer-spike protein rupture force is a parameter to measure. An additional potential advantage to mechanical transduction is that it may reduce non-specific binding and detect transient interactions. ACE2-spike complexes with similar affinity do show rupture forces of 57 pN when using 800 pN/s loading rates. It is estimated that each motor generates approximately 100 pN of force, but this force is dampened because there is significantly lower density of DNA and RNA on the motor and chip, respectively. Calculations suggests that lowering the magnitude of force generated by the motor can lead to enhanced biosensor performance indicating that a single virus particle presenting 20-40 copies of trimeric spike protein can lead to motor stalling. Interestingly, when GFP-tagged VLPs were used in Rolosense, a population of stalled motors colocalized with single VLPs suggesting that Rolosense motors can respond and report on single SARS-CoV-2 virions.

Experiments indicate that in artificial saliva one can detect up to 10³ copies/mL of SARS-CoV-2 WA-298 1, B.1.617.2, and the variant of concern BA.1. To validate the specificity of the Rolosense assay, cross-reactivity with other respiratory viruses such as the seasonal common cold 300 viruses, HCoV OC43 and 229E, as well as influenza A was tested. A distinguishable effect on Rolosense response was not observe. A key advantage of Rolosense is the ability to multiplex and detect multiple respiratory viruses in the same assay. This capability is important for point-of-care diagnostics and in minimizing false positive results due to similar symptoms caused by other respiratory viruses. By encoding different virus specific DNA motors through size and refractive index one can distinguish between SARS-CoV-2 and influenza A in one “pot.” With the aim of enabling the key steps for a point-of-care diagnostic, Rolosense motors and chip can be used to conveniently detect SARS-CoV-2 using a smartphone and a magnifying lens as the reader. The assay was performed using a rapid, about 15 min readout. The assay is suitable for exhaled breath condensate testing. An LoD of 10³ copies/mL demonstrated for the B.1.617.2 and BA.1 variants which is comparable to that of lateral flow assays like the BinaxNOW™ COVID-19 Ag Card which have an LoD of 10⁵ copies/mL for the BA.1 variant. Rolosense takes advantage of multivalent binding which may contribute to LoD that is better than that of monomeric assays like LFA. Another strength of Rolosense is that it is highly modular and any whole virion that displays many copies of a target can be detected using appropriate aptamers. Also, multiplexed detection of SARS-CoV-2 and influenza A can, in principle, be scaled up to include a panel of viral targets. One could create two or more of uniquely encoded motors. Multiplexed Rolosense is useful in clinical applications as the assay is rapid and can be conducted conveniently without the need for a dedicated PCR instrument.

TABLE 1
shows DNA-based motors and chips with DNA aptamers

| ID | Sequences (5′-3′) | |
|---|---|---|
| DNA Anchor | /5AmMC6/GAGAGAGATGGGTGCTTT | SEQ ID |
| | TTTTTTTTTTTT/3ThiolMC3-D/ | NO: 1 |
| RNA/DNA | GCACCCATCTCTCTCCCCCCrCrUrG | SEQ ID |
| | rUrGrArUrUrGrArUrUrArCrU/ | NO: 2 |
| | 3Cy3Sp/ | |
| DNA Leg | /5Hexynyl/TTTTTTTTTTTTTTTAG | SEQ ID |
| | TAATCAATCACAG | NO: 3 |
| Particle | /5Hexynyl/TTTTTCGCGGTCATTG | SEQ ID |
| Aptamer 1 | TGCATCCTGACTGACCCTAAGGTGCG | NO: 4 |
| | AACATCGCCCGCG | |
| Surface | CGCGGTCATTGTGCATCCTGACTGAC | SEQ ID |
| Aptamer 1 | CCTAAGGTGCGAACATCGCCCGCGGC | NO: 5 |
| | ACCCATCTCTCTC | |
| Particle | /5Hexynyl/TTTTTGGGAGAGGAGG | SEQ ID |
| Aptamer 2 | GAGATAGATATCAACCCATGGTAGGT | NO: 6 |
| | ATTGCTTGGTAGGGATAGTGGGCTTG | |
| | ATGTTTCGTGGATGCCACAGGAC | |
| Surface | GGAGAGGAGGGAGATAGATATCAACC | SEQ ID |
| Aptamer 2 | CATGGTAGGTATTGCTTGGTAGGGAT | NO: 7 |
| | AGTGGGCTTGATGTTTCGTGGATGCC | |
| | ACAGGACGCA CCC ATCTCTCTC | |
| Part Apt 3 | /5Hexynyl/TTTTTCCCATGGTAGGT | SEQ ID |
| | ATTGCTTGGTAGGGATAGTGGG | NO: 8 |
| Surf Apt 3 | CCCATGGTAGGTATTGCTTGGTAGGG | SEQ ID |
| | ATAGTGGGGCACCCATCTCTCTC | NO: 9 |
| Part Apt 4 | /5Hexynyl/TTTTTACGCCAAGGTGT | SEQ ID |
| | CACTCCGTAGGGTTTGGCTCCGGGCC | NO: 10 |
| | TGGCGTCGGTCG CGAAGCATCT CC | |
| | TTGGCGT | |
| Surf Apt 4 | ACGCCAAGGTGTCACTCCGTAGGGTT | SEQ ID |
| | TGGCTCCGGGCCTGGCGTCGGTCGCG | NO: 11 |
| | AAGCATCTCCTTGGCGTGCACCCATC | |
| | TCTCTC | |
| InfluenzaA | /5Hexynyl/TTTTTGGCAGGAAGACA | SEQ ID |
| Part Apt | AACAGCCAGCGTGACAGCGACGCGTA | NO: 12 |
| | GGGACCGGCATCCGGGGGTGGTCTGT | |
| | GGTGCTGT | |
| InfluenzaA | GGCAGGAAGACAAACAGCCAGCGTGA | SEQ ID |
| Surf Apt | CAGCGACGCGTAGGGACCGGCATCCG | NO: 13 |
| | CGGGTGGTCTGTGGTGCTGTGCA CC | |
| | C ATC TCT CTC | |

DNA aptamers with affinity for spike protein (S1) that were prepared. Aptamers as virus binding ligands have several advantages such as ease of storage, long-term stability, and a smaller molecular weight. Amine modified motors were functionalized and coated with a binary mixture of both the DNA leg and aptamer 1. The planar Rolosense chip was modified with a binary mixture of Cy3-labeled RNA fuel and aptamer 1. The oligonucleotides were tethered to the surface by hybridization to a monolayer of 15mer ssDNA, referred to as the DNA anchor.

Tuning the ratio of the DNA legs/RNA fuel to the aptamer was critical as there is a tradeoff between multivalent avidity to the virion and efficient motor motion. For example, high densities of aptamer lead to efficient virus binding but hamper processive motion. Conversely, low aptamer densities diminish virus binding, but enhance motor speed and processivity. Accordingly, different ratios of aptamer/DNA leg on the particle were screened. Aptamer/RNA fuel on the chip were used to measure motor net displacement over a 30 min time window. The introduction of aptamer at 10% density or greater on the particle led to a significant reduction in motor displacements. Also, motor distance was more sensitive to aptamer density on the spherical particle compared to that of the planar surface. An optimal aptamer density was 10% for the particle and 50% for the planar surface, as these motors showed 1.95+/−0.97 μm net displacement over a 30 min time window compared to the no aptamer control in which the motors traveled 2.56+/−1.17 μm in 30 mins. Based on these results, all subsequent experiments were conducted using motors, and chips modified with 10%, and 50% aptamer density, respectively.

Assays were tested using GFP-tagged virus-like particles (VLPs) expressing a SARS-CoV-2 trimeric spike protein. non-infectious HIV-1 and SARS-CoV-2 spike (D614G mutation) virus-like particles VLPs were used. As a control to test for cross reactivity, GFP-tagged HIV-1 particles that lacked spike protein (bald VLPs) were used. The motor surface was functionalized with 10% aptamer 1 and chip surface with 50% aptamer 1. The VLPs were incubated with the aptamer functionalized DNA-based motors in 1× PBS (phosphate-buffered saline) for 30 mins at room temperature. After 30 mins, the DNA-based motors were washed via centrifugation (15,000 rpm, 1 min) and then added to the Rolosense chip that was also coated with the same aptamer. In the presence of RNase H the DNA-based motors incubated with the spike VLPs remained stalled on the surface. The VLPs were likely sandwiched between the DNA-based motor and the chip surface, and this binding led to a stalling force that halted motion. In contrast, DNA-based motors incubated with the bald VLPs lacking the spike protein translocated on the surface which was expected because the bald VLPs do not bind to the aptamers. This was confirmed by optical and fluorescence microscopy. Motors incubated with the bald VLPs displayed micron-length depletion tracks in the Cy3-RNA monolayer. The lack of fluorescence signal in the GFP channel indicates that there was minimal binding of bald VLPs to the motors. On the contrary, motors incubated with spike VLPs did not display Cy3-RNA depletion tracks and the GFP fluorescence channel showed puncta colocalized with the stalled motors confirming that the stalling was due to spike VLPs binding Brightfield real-time particle tracking also validated this conclusion. Long trajectories and net displacements greater than 1.5 μm was observed for motors incubated with bald VLPs. The spike VLP incubated motors, on the other hand, displayed short trajectories and sub 1 μm net displacements. Control motors without VLPs showed greater displacements than that of the bald VLP samples, likely due to non-specific bald VLPs binding. These results demonstrate that the Rolosense design and mechanism for viral detection is valid.

It is contemplated that Rolosense is not unique to aptamers and virtually any virus binding ligand could be used for viral sensing. However, in preliminary screens with two commercial antibodies, motor stalling was found with bald VLPs suggesting issues with specificity. Efforts were directed to screening across different aptamers reported to display high affinity and specificity for SARS-CoV-2 S1.

Specifically, aptamer 1,

(SEQ ID NO: 14)
CGCGGTCATTGTGCATCCTGACTGACCCTAAGGTGCGAACATCGC
CCGCG,
aptamer 2,
(SEQ ID NO: 15)
GGAGAGGAGGGAGATAGATATCAACCCATGGTAGGTATTGCTTGG
TAGGGATAGTGGGCTTGATGTTTCGTGGATGCCACAGGAC,

and aptamer 3,

CCCATGGTAGGTATTGCTTGGTAGGGATAGTGGG (SEQ ID NO: 16) have reported KD values in the low nanomolar range for S1. Using motors and surfaces functionalized with each of these aptamers (10% motor, and 50% chip), aptamer 3 was the most sensitive and specific for Rolosense.

### Detecting SARS-CoV-2 in Artificial Saliva

Experiments were performed to validate the Rolosense assay using authentic SARS-CoV-2 virus that was UV-inactivated. The Washington (WA-1) strain was tested. For these sets of experiments, the virus was spiked into artificial saliva and the Rolosense method was performed for viral readout. Experiments were performed to determine whether the motors and the Rolosense assay could tolerate the artificial saliva matrix since it contains mucins and divalent ions such as calcium that may interfere with the assay. Motors were suspended in artificial saliva for 30 min and then added to the aptamer-decorated chip for readout. Motion was not affected by the artificial saliva matrix and the motors displayed long trajectories with the addition of RNase H enzyme and net displacement (2.20 μm+/−1.38 μm) was comparable to controls performed in 1× PBS (2.97 μm 142+/−1.40 μm). Once the assay was validated in artificial saliva, motors functionalized with 10% of aptamer 3 were incubated with 10⁸ copies/mL of SARS-CoV-2 WA for 30 mins at room temperature. After 30 mins, the DNA-based motors were washed via centrifugation (15,000 rpm, 1 min) and then added to the Rolosense chip presenting aptamer 3. In the presence of RNase H the motors remained stalled on the surface and did not display depletion tracks. Control motors without virus displayed long depletion tracks in the Cy3-RNA channel. Brightfield particle tracking confirmed these results. Particle trajectories for SARS-CoV-2 WA-1 condition were compared to the long trajectories displayed by motors without any virus. In a control experiment, wherein the surface aptamer was withheld, aptamer presenting motors incubated with 10⁷ copies/mL of SARS-CoV-2 WA-1 displayed long net displacements and depletion tracks in the Cy3-RNA channel. This confirmed that the stalling observed in the presence of virus is due to virus particles bridging the aptamers on the bead to the aptamers on the chip. To optimize workflow of the Rolosense assay, experiments were performed to determine whether one could forego the washing step following motor incubation with virus. Running the assay without the wash step does not degrade the integrity of the chip as the RNA on the surface remained intact. Similar net displacements were also observed between the motors with and without wash when incubated with 10⁷ copies/mL of WA-1 virus. In addition, experiments were performed to determine whether decreasing the virus sample incubation time affects the performance of Rolosense. Decreasing the incubation time with the motors down to 10 minutes does not impact the performance of the Rolosense assay as many of the motors remained stalled.

Experiments were performed to determine the limit of detection (LoD) of the Rolosense assay in artificial saliva. In triplicate experiments a LoD of about 10⁴ copies/mL for the Washington strain 1 (WA-1) was demonstrated. The Rolosense assay with other SARS-167 CoV-2 variants such as Delta (B.1.617.2) and Omicron (BA.1) spiked in artificial saliva. The Rolosense assay showed a sensitive response to both B.1.617.2 and BA.1 with an LoD of about 10³-10⁴ copies/mL. The LoD for the B.1.617.2 and WA-1 was greater than that for the BA.1 variant which was expected given that aptamer 3 was selected using S1 of the initial Wuhan strain. Interestingly, the mutations in S1 for the B.1.617.2 strain primarily led to an increase in the net positive charge of the protein which likely aids in enhancing binding to a negatively charged aptamer. The LoD for the BA.1 strain is weaker, there is an increased number of mutations in this most recent variant. Rolosense demonstrates an LoD that is akin to that of typical LFAs but using a DNA motor.

To test for cross-reactivity and specificity the response of the motors incubated with other respiratory viruses such as the seasonal common cold viruses, HCoV OC43 178 and 229E, as well as the influenza A virus were remeasured. These respiratory viruses present similar symptoms as the SARS-CoV-2 virus and thus it is important to distinguish between them. These samples were prepared in a similar manner to that of the SARS-CoV-2 variants and spiked in artificial saliva to run the Rolosense assay. The motors displayed high specificity and responded with motion to HCoV OC43, HCoV 229E, and Influenza A which is in direct contrast to the stalling observed in the presence of SARS-CoV-2 viruses. This data confirms that the Rolosense assay exhibits a sensitive and specific response to the SARS-CoV-2 virus which is ultimately the result of the sensitivity and specificity of aptamer 3 to its SARS-CoV-2 target.

### Multiplexed Detection of SARS-CoV-2 and Influenza A viruses

Given the need for distinguishing between a variety of respiratory viruses, experiments were performed to determine whether Rolosense can detect other viruses such as the influenza A virus. An influenza A motor was created by modifying it with 10% of influenza A aptamer, with the chip presenting 50% aptamer. Following the protocol for SARS-CoV-2, the motors were incubated with different concentrations of influenza A virus spiked in 1× PBS for 30 min. Although the motors stalled in the presence of high concentrations of influenza A virus such as 10¹⁰ copies/mL, the assay performed poorly in detecting low copy numbers. To address this issue, the 1× PBS solution was supplemented with 1.5 mM Mg⁺² since divalent cations aid in secondary structure formation of aptamers. The assay improved with the addition of Mg⁺² and one is able to detect as low as 10⁴ copies/mL of influenza A virus using this aptamer. This suggests potential for Rolosense to detect influenza A infections, as a typical swab of patients with influenza yield about 10⁸ genome copies/ml as estimated by PCR.

It is contemplated that these methods can be used for multiplexed detection of SARS-CoV-2 and influenza A in the “same pot.” To achieve this, two different motors were used: 5 μm silica bead functionalized with influenza A aptamer and 6 μm polystyrene bead functionalized with aptamer 3. Size refractive index of different particles can be used to optically encode each motor in a label free manner using brightfield contrast. The chip was functionalized with 25% influenza A aptamer and 25% aptamer 3. When the influenza A motors (5 μm silica) and SARS-CoV-2 motors (6 μm polystyrene) were not incubated with virus, they responded with motion in the presence of RNase H. Long depletion tracks were observed in the Cy3 channel for both motors and analysis from brightfield particle tracking of over 300 motors resulted in net displacements of 2.88 μm+/−2.00 μm and 2.68 μm+/−1.83 μm for the influenza A and SARS-CoV-2 motors, respectively. In the same tube, both motors were then incubated with 10¹⁰ copies/mL of the influenza A virus (in 1× PBS with 1.5 mM Mg⁺²) for 30 mins at room temp. As a result, the influenza A motors remained stalled on the chip while the SARS-CoV-2 motors were free to move in the presence of RNase H. Depletion tracks were not observed in the Cy3 channel for the influenza A motor, but the SARS-CoV-2 motors formed long depletion tracks. Brightfield particle tracking confirmed this result as the net displacement of the influenza A virus decreases, compared to no virus, and the SARS-CoV-2 motors exhibited an average net displacement of 2.60+/−2.23 μm. The motors were also incubated with 10⁷ copies/mL of SARS-CoV-2 WA-1 in 1× PBS with 1.5 mM Mg⁺². In this condition, no tracks were observed for the SARS-CoV-2 motor but the influenza A motors formed long tracks. The average net displacement of the influenza A motors was 1.97 μm+/−1.84 μm compared to 0.81+/−0.77 μm for the SARS-CoV-2 motor. As a control, the motors were incubated with both viruses, and they remained stalled on the chip. All in all, using different size beads with different optical intensities one can multiplexed viral detection on the same chip.

### Detecting SARS-CoV-2 by Smartphone Readout

Smartphone based sensors have captured the interest of the public health community because of their global ubiquity and their ability to provide real-time geographical information of infections. Rolosense is highly amenable to smartphone readout because smartphone cameras modified with an external lens can easily detect the motion of micron-sized motors. As a proof-of-concept, a smartphone (iPhone 13) was used to detect the motion of Rolosense motors exposed to artificial saliva spiked with SARS-CoV-2. A simple smartphone microscope set up (Cellscope™) was used which includes an LED light source and 10× magnification lens. For these experiments, DNA motors and chip were functionalized with aptamer 3. SARS-CoV-2 WA-1 and 236 B.1.617.2 stocks were serially diluted in artificial saliva. The DNA motors were added to these known concentrations of virus and the samples were incubated 30 mins at room temperature. Following incubation in artificial saliva, the samples were added to the Rolosense chip and imaged for motion via smartphone. The smartphone analyzed timelapse imaging data matched that of high-end microscopy analysis, and in 15 mins timelapse videos one could detect the presence of SARS-CoV-2 in artificial saliva with an LoD of about 10³ copies/mL. More sensitive detection of SARS-CoV-2 B.1.617.2 2 was observed than WA-1 using aptamer 3. These experiments indicate the feasibility of label free SARS-CoV-2 sensing using smartphone camera.

### Detecting SARS-CoV-2 in Breath Condensate Generated Samples

Exhaled breath offers the most non-invasive and accessible biological markers for diagnosis. Experiments were performed to evaluate performance by using exhaled breath condensate as the sensing medium since. Exhaled breath is cooled and condensed into a liquid phase and consists of water-soluble volatiles as well as non-volatile compounds. Breath condensate was collected and mixed with our motors without any virus to test whether Rolosense can tolerate breath condensate as the medium (FIG. 5A and 5B). Experiments indicate that breath condensate did not affect the robustness of the assay as motors without virus displayed comparable net displacements to motors diluted in 1× PBS. Breath condensate displayed little DNase and RNase activity, as indicated by the high fluorescence signal of the RNA monolayer.

To determine the LoD of SARS-CoV-2 sensing in breath condensate, samples were prepared in a similar manner as in artificial saliva. The DNA-based motors and chip were functionalized with aptamer 3. B.1.617.2 stocks were serially diluted in collected breath condensate. The DNA-based motors were incubated with the virus samples in breath condensate for 30 mins at room temperature. After incubation, the samples were added to the Rolosense chip and imaged. From 264 brightfield particle tracking, we demonstrate an LoD of approximately 10³ copies/mL for SARS-CoV-2 B.1.617.2. The LoD is not affected when using breath condensate as the virus sensing matrix. Aptamer 4 is capable of targeting the S1 subunit of the spike protein of the BA.1 variant with high affinity. Therefore, to increase sensitivity in detecting the SARS-CoV-2 BA.1 variant, motor and chip surfaces were functionalized with this BA.1 specific aptamer. With aptamer 4, on can detect as low as 10³ copies/mL of the BA.1 variant and possibly as low as 10² copies/mL. These LoDs are highly promising as studies indicate that at early stages of infection with SARS-CoV-2 273 the estimated breath emission rate is 10⁵ virus particles/min, which 5 suggests that 1 min of breath condensate collection will provide sufficient material for accurate SARS-CoV-2 detection.

1. A method of detecting the presence of a microbe in a sample comprising,
contacting a sample suspected of containing microbe and a spherical particle comprising a coating of single stranded DNA and an aptamer that specifically binds a microbial biomolecule providing a sample exposed spherical particle;
providing a planar substrate comprising a coating of single stranded RNA and an aptamer that specifically binds the microbial biomolecule;
placing the sample exposed spherical particle on the surface of the planar substrate in the presence of RNase H such that the particle moves on the surface of the substrate; and
measuring the movement of the sample exposed spherical particle on the surface of the planar substrate providing a sample exposed movement value;
comparing the sample exposed movement value to a reference movement value correlated to the movement of the spherical particle on the surface of the planar substrate in the presence of RNase H and in the absence of the sample and
detecting the presence of a microbe in a sample if the movement value is less than the reference movement value.

2. The method of claim 1, wherein the aptamer to the single stranded DNA on the spherical particle is about 10% or less by weight.

3. The method of claim 1, wherein the aptamer to the single stranded RNA on the planar substrate is about 50% or less by weight.

4. The method of claim 1 wherein the aptamer comprises the nucleotide sequence of
(SEQ ID NO: 16)
CCCATGGTAGGTATTGCTTGGTAGGGATAGTGGG

5. The method of claim 1, wherein the aptamer comprises the nucleotide sequence of
(SEQ ID NO: 17)
ACGCCAAGGTGTCACTCCGTAGGGTTTGGCTCCGGGCCTGGCGTC
GGTCGCGAAGCATCTCCTTGGCGT

6. The method of claim 1, the aptamer comprises the nucleotide sequence of
(SEQ ID NO: 18)
GGCAGGAAGACAAACAGCCAGCGTGACAGCGACGCGTAGGGACCG
GCATCCGCGGGTGGTCTGTGGTGCTGTGCA.

7. The method of claim 1, wherein the microbe is a coronavirus.

8. The method of claim 1, wherein the microbe is influenza.

9. The method of claim 1, wherein detecting the presence of a microbe in a sample if the movement value is less than the reference movement value is by use of a video camera and computer.

10. The method of claim 1, wherein detecting the presence of a microbe in a sample if the movement value is less than the reference movement value is by quantifying a lower displacement or in a more restricted area that the particle would move when compared to a value determined in the absence of the microbial biomolecule in the sample.

11. A rolling particle system comprising,
a spherical particle comprising a coating of single stranded DNA and an aptamer that specifically binds the microbial biomolecule; and
a planar substrate comprising a coating of single stranded RNA and an aptamer that specifically binds the microbial biomolecule.

12. The rolling particle system of 11, wherein the aptamer to the single stranded DNA on the spherical particle is about 10% or less by weight.

13. The rolling particle system of 11, wherein the aptamer to the single stranded RNA on the planar substrate is about 50% or less by weight.

14. The rolling particle system of 11, wherein the microbe is coronavirus.

15. The rolling particle system of claim 11, wherein the microbe an influenza virus.

16. The rolling particle system of 11, wherein the aptamer is
(SEQ ID NO: 16)
CCCATGGTAGGTATTGCTTGGTAGGGATAGTGGG.

17. The rolling particle system of 11, wherein the aptamer is
(SEQ ID NO: 17)
ACGCCAAGGTGTCACTCCGTAGGGTTTGGCTCCGGGCCTGGCGTC
GGTCGCGAAGCATCTCCTTGGCGT

18. The rolling particle system of 11, wherein the aptamer is
(SEQ ID NO: 18)
GGCAGGAAGACAAACAGCCAGCGTGACAGCGACGCGTAGGGACCG
GCATCCGCGGGTGGTCTGTGGTGCTGTGCA.


SEQUENCE LISTING (18 sequences)

SEQ ID NO: 1 - DNA, 30 bp, synthetic construct
  source 1..30: mol_type = other DNA
gagagagatgggtgcttttttttttttttt

SEQ ID NO: 2 - RNA, 35 nt, synthetic construct
  source 1..35: mol_type = other RNA
gcacccatctctctccccccctgtgattgattact

SEQ ID NO: 3 - DNA, 30 bp, synthetic construct
  source 1..30: mol_type = other DNA
tttttttttttttttagtaatcaatcacag

SEQ ID NO: 4 - DNA, 55 bp, synthetic construct
  source 1..55: mol_type = other DNA
tttttcgcggtcattgtgcatcctgactgaccctaaggtgcgaacatcgcccgcg

SEQ ID NO: 5 - DNA, 65 bp, synthetic construct
  source 1..65: mol_type = other DNA
cgcggtcattgtgcatcctgactgaccctaaggtgcgaacatcgcccgcggcacccatctctctc

SEQ ID NO: 6 - DNA, 91 bp, synthetic construct
  source 1..91: mol_type = other DNA
tttttgggagaggagggagatagatatcaacccatggtaggtattgcttggtagggatagtgggcttgatgtttcgtggatgccacaggac

SEQ ID NO: 7 - DNA, 100 bp, synthetic construct
  source 1..100: mol_type = other DNA
ggagaggagggagatagatatcaacccatggtaggtattgcttggtagggatagtgggcttgatgtttcgtggatgccacaggacgcacccatctctctc

SEQ ID NO: 8 - DNA, 39 bp, synthetic construct
  source 1..39: mol_type = other DNA
tttttcccatggtaggtattgcttggtagggatagtggg

SEQ ID NO: 9 - DNA, 49 bp, synthetic construct
  source 1..49: mol_type = other DNA
cccatggtaggtattgcttggtagggatagtggggcacccatctctctc

SEQ ID NO: 10 - DNA, 74 bp, synthetic construct
  source 1..74: mol_type = other DNA
tttttacgccaaggtgtcactccgtagggtttggctccgggcctggcgtcggtcgcgaagcatctccttggcgt

SEQ ID NO: 11 - DNA, 84 bp, synthetic construct
  source 1..84: mol_type = other DNA
acgccaaggtgtcactccgtagggtttggctccgggcctggcgtcggtcgcgaagcatctccttggcgtgcacccatctctctc

SEQ ID NO: 12 - DNA, 77 bp, synthetic construct
  source 1..77: mol_type = other DNA
tttttggcaggaagacaaacagccagcgtgacagcgacgcgtagggaccggcatccgcgggtggtctgtggtgctgt

SEQ ID NO: 13 - DNA, 87 bp, synthetic construct
  source 1..87: mol_type = other DNA
ggcaggaagacaaacagccagcgtgacagcgacgcgtagggaccggcatccgcgggtggtctgtggtgctgtgcacccatctctctc

SEQ ID NO: 14 - DNA, 50 bp, synthetic construct
  source 1..50: mol_type = other DNA
cgcggtcattgtgcatcctgactgaccctaaggtgcgaacatcgcccgcg

SEQ ID NO: 15 - DNA, 85 bp, synthetic construct
  source 1..85: mol_type = other DNA
ggagaggagggagatagatatcaacccatggtaggtattgcttggtagggatagtgggcttgatgtttcgtggatgccacaggac

SEQ ID NO: 16 - DNA, 34 bp, synthetic construct
  source 1..34: mol_type = other DNA
cccatggtaggtattgcttggtagggatagtggg

SEQ ID NO: 17 - DNA, 69 bp, synthetic construct
  source 1..69: mol_type = other DNA
acgccaaggtgtcactccgtagggtttggctccgggcctggcgtcggtcgcgaagcatctccttggcgt

SEQ ID NO: 18 - DNA, 75 bp, synthetic construct
  source 1..75: mol_type = other DNA
ggcaggaagacaaacagccagcgtgacagcgacgcgtagggaccggcatccgcgggtggtctgtggtgctgtgca